(19) United States
(12) Patent Application Publication
(10) Pub. No.: US 2005/0176140 A1
(43) Pub. Date: Aug. 11, 2005

(54) Method and apparatus for cell culture using a two liquid phase bioreactor

(76) Inventors: Daniel J. Benedict, Chicago, IL (US); Lorna S. Mosse, Chicago, IL (US)

Correspondence Address: DANIEL J. BENEDICT, 3037 S. PRINCETON AVE, 3R, CHICAGO, IL, 60616, US

(21) Appl. No.: 11/055,452

(22) Filed: Feb. 10, 2005

Related U.S. Application Data
(60) Provisional application No. 60/542,971, filed on Feb. 10, 2004.

(51) Int. Cl. C12N 5/08

(52) U.S. Cl. 435/366; 435/404

(57) ABSTRACT

Advanced Bioreactor Cell Culture Technology presents a method of cell culturing and bioprocessing incorporating molecular biology techniques, advanced process control methodology, and a process control interface applied to a two liquid phase cell culture bioreactors to proliferate, grow, and expand non-differentiated precursor cells, embryonic stem (ES) cells, endocrine progenitor cells, pancreatic progenitor cells, pancreatic stem cells, pancreatic duct epithelial cells, nestin-positive islet-derived progenitor cells (NIPs), or pluripotent non-embryonic stem (PNES) cells in the bioreactor, and influence, stimulate, and induce the non-differentiated precursors and progenitors into fully differentiated beta cell phenotypes; including microprocessor control of cell culture process variables and data acquisition during bioprocessing. The invention may be applied to precursors and progenitor cells either transgenic or non-transgenic derived from animals and mammals.

(This application claims priority under 35 USC 119(e). This application is a continuation of and claims benefit of U.S. PTO Ser. No. 60/542,971.)

### CROSS-REFERENCE TO RELATED APPLICATIONS

“Not Applicable”

### STATEMENT REGARDING FEDERALLY SPONSORED RESEARCH OR DEVELOPMENT

“Not Applicable”

### REFERENCE TO A MICROFICHE APPENDIX

“Not Applicable”

### FIELD OF THE INVENTION

The present invention relates to the application of culturing, proliferation, and growth of mammalian cells and animal cells in two liquid phase bioreactors. The invention presents Advanced Bioreactor Cell Culture Technology, a method of cell culturing and bioprocessing incorporating molecular biology techniques, advanced process control methodology, and a process control interface applied to two liquid phase cell culture bioreactors to culture fully differentiated cells, or culture, proliferate, and grow non-differentiated precursor cells, embryonic stem (ES) cells, progenitor cells, stem cells, pluripotent non-embryonic stem (PNES) cells, and influence, stimulate, and induce the non-differentiated precursors and progenitors into fully differentiated cells of different phenotypes; including microprocessor control of cell culture process variables and data acquisition during bioprocessing. The invention may be uniformly applied to precursors such as ES cells, progenitor cells, stem cells, pluripotent non-embryonic stem (PNES) cells, or fully differentiated cells, derived from animals and mammals either transgenic or non-transgenic. The invention may be uniformly applied to anchorage dependent cells and non-anchorage dependent cells derived from animals and mammals either transgenic or non-transgenic.

### BACKGROUND OF THE INVENTION

The islets of Langerhans contain insulin producing beta cells. These may be optimally harvested from pancreases, either human or porcine using Advanced Islet Separation Technology. This islet processing technology can yield sufficient numbers of uniform quality healthy islets for transplantation, or encapsulation within a viable artificial pancreas. Sufficient numbers of uniform quality healthy islets provide one means to alleviate the suffering of large numbers of individuals with Type I diabetes mellitus.

Different precursor cells may be induced to differentiate and become beta cell phenotypes. Another method to ameliorate large numbers of individuals with Type I diabetes mellitus is to culture and grow large quantities of uniform quality healthy beta cell phenotypes suitable for transplantation or encapsulation. This may be accomplished by culturing, proliferating, expanding, growing, and inducing differentiation in animal or mammalian precursor cells, ES cells, endocrine progenitor cells, pancreatic progenitor cells, pancreatic stem cells, pancreatic duct epithelial cells, nestin-positive islet-derived progenitor cells (NIPs), or pluripotent non-embryonic stem (PNES) cells in a bioreactor. There is controversy in employment of ES cells; yet, precursor cells, endocrine progenitor cells, pancreatic duct epithelial cells, pancreatic progenitor cells, pancreatic stem cells, nestin-positive islet-derived progenitor cells (NIPs), or pluripotent non-embryonic stem (PNES) cells developed into fully differentiated cells present no controversy for transplantation or encapsulation. The important bioprocessing objective in bioreactor cell culturing is to produce large quantities of viable functional and potent beta cell phenotypes for transplantation or encapsulation.

One crucial requirement for successful cell culturing, cell proliferation, abundant cell growth, and differentiation is cell specific culture media. There exist many cell specific culture media formulations for the diverse types of cells. Cell specific culture media are constantly being refined to influence, stimulate, and induce cellular differentiation in precursor and progenitor cells by the addition of specific biochemical agents consisting of growth factors, hormones, enzymes, peptides, and transcription factors. Controlling important cell culture process variables in a bioreactor is another crucial cell culture requirement that influences cellular growth dynamics. Important and crucial cell culture process variables include the cell culture media temperature (T), the cell culture media hydrogen (pH) ion concentration, the cell culture media dissolved oxygen (DO) concentration, the cell culture media dissolved carbon dioxide (CO₂) concentration, the cell culture media nitric oxide (NO) concentration, the cell culture media endotoxin (E) concentration, the cell culture media antibiotic (A) concentration, and the cell culture media agitator (S) speed. Apoptosis inhibition is another crucial cell culture requirement and may be accomplished with biochemical agents consisting of free radical scavenging enzymes, hormones, amino acids, and antibiotics added to cell culture media. Inducing cellular differentiation is another crucial cell culture requirement in producing differentiated beta cell phenotypes and may be accomplished with biochemical agents consisting of growth factors, hormones, and transcription factors added to cell culture media.

Endotoxin is associated with a number of cellular events including cell activation with subsequent cytokine secretion and programmed cell death, apoptosis. Endotoxin exposure is postulated to cause a loss of functionality in pancreatic islets, Vargas et al., Transplantation, 65(5):722-727, Mar. 15, 1998, incorporated herein by reference. They have demonstrated that biochemical factors in supernatants were able to induce certain inflammatory cytokines in islets. Jahr et al., J. Mol. Medicine (Berlin), 77(1):118-120, January 1999, incorporated herein by reference, suggest that endotoxin-induced early inflammatory reactions may inhibit the function and survival of cells or cell aggregates. Eckhardt et al., J. Mol. Medicine (Berlin), 77(1):123-125, January 1999, incorporated herein by reference, have determined that islet cell functionality increased in endotoxin free conditions. Endotoxin and accompanying cellular reactions cause cellular non-function. Endotoxin is detrimental to cell culturing, cell proliferation, cell growth, and cellular differentiation in a bioreactor.

Nitric oxide and its metabolites are known to cause cellular death and apoptosis from nuclear damage U.S. Pat. No. 5,834,005, A. Usula, Nov. 10, 1998, incorporated herein by reference. Nitric oxide is a recognized multifunctional mediator that is produced by and acts on various cells, and participates in inflammatory and autoimmune-mediated tissue destruction, U.S. Pat. No. 5,919,775, Amin et al., Jul. 6, 1999, incorporated herein by reference. The group of enzymes known as nitric oxide synthases catalyzes nitric oxide production. Nitric oxide synthase (NOS) is expressed in mammalian cells. Utilizing cofactors in the presence of oxygen, it catalyzes the mixed functional oxidation of L-arginine to L-citrulline and nitric oxide, by removing a guanidino nitrogen from L-arginine to form nitric oxide. Interleukin-1 (IL-1) has been shown to induce the expression of the cytokine inducible isoform of nitric oxide synthase in pancreatic islets. The production of nitric oxide has been proposed to be the effector molecule that mediates IL-1's inhibitory effects on islet function, U.S. Pat. No. 5,837,738, Williamson et al, Nov. 17, 1998, herein incorporated by reference.

The deleterious effects of nitric oxide on islet cells can be alleviated by a variety of means. Inhibitors of nitric oxide synthase have been identified. Nitric oxide synthase (NOS) and subsequent nitric oxide can be inhibited by derivatives of L-arginine, the natural substrate of nitric oxide synthase. These include methyl-, dimethyl-, or amino-substituted guanidines. These inhibitory compounds are also chemically known as aminoguanidinie, N,N′-diaminoguanidine, methylguanidine and 1,1-dimethylguanidine (U.S. Pat. No. 5,837,738 and U.S. Pat. No. 5,919,775, both previously incorporated herein by reference). Nitric oxide production can also be inhibited by 2,4-diamino-6-hydroxypyrimidine, a compound that interferes with the activity of a cofactor of inducible NOS. Antibiotic tetracycline also inhibits nitric oxide synthase, thus preventing the formation of nitric oxide, as do doxycycline, and minocycline, a semi-synthetic tetracycline (U.S. Pat. No. 5,919,775, previously incorporated herein by reference). Nitric oxide can also be inhibited by nitric oxide scavengers such as cysteine, and other sulfated compounds such as dextran, heparin, and cystine (U.S. Pat. No. 5,834,005, previously incorporated herein by reference). Cysteine, dextran, heparin, and cystine also inhibit nitric oxide formation that results from relative states of islet hypoxia. An overexpression of nitric oxide synthase (NOS)-2, is responsible for the high-output nitric oxide synthesis in cells stimulated with pro-inflammatory cytokines, Castrillo, A. et al., Diabetes: 49, 209-217, 2000, incorporated herein by reference. Insulin like growth factor-1 (IGF-1) exerts an inhibitory effect on the expression of NOS-2 as well as on the nitric oxide and cytokine dependent appptosis in isolated beta-pancreatic cells. Nitric oxide inhibition and scavenging improves survival of precursor and progenitor cells and improves secretory function in fully differentiated beta cell phenotypes.

Reactive oxide species (ROS), oxide and hydroxyl free radicals are known to cause nuclear damage leading to cellular death and apoptosis. Three main intracellular ROS scavenging enzymes are superoxide dismutase (SOD), catalase, and glutathione peroxidase (GSHpx), Cederberg et al., Diabetes 49:101-107, 2000, incorporated herein by reference. All three differ in subcellular distribution and type of catalyzed reaction. SOD catalyzes the conversion of superoxide ions into oxygen and hydrogen peroxide and exists in a cytoplasmatic form (CuZn-SOD) and a mitochondrial form (Mn-SOD). Catalase is distributed mainly to the peroxisomes and catalyzes the decomposition of hydrogen peroxide into water and oxygen. GSHpx reduces hydrogen peroxide to water using glutathione (GSH), which is in turn oxidized to oxidized glutathione (GSSG).

L-ergothioneine is a powerful antioxidant, Akanmu, D. et al., Arch. Biochem. Biophys., 288:10-16, 1991, incorporated herein by reference. It neutralizes hydroxyl radicals and hypochlorous acid. Unlike other thiol antioxidants, it does not induce lipid peroxidation in the presence of ferric acids. It selectively increases the antioxidant activity enzymes selenium dependent glutathione peroxidase (Se-GPx), glutathione reductase (GR), and magnesium super oxide dismutase (Mn-SOD), Spolarics, Z., Am J Physiol—Gastrointest Liver Physiol, 270, Issue 4, 660-G666, 1996, and Dahl, T. A. et al., Photeochem. and Photobiol, 47, 357-362, 1988, both incorporated herein by reference. Mitochondria are subcellular organelles present in all oxygen-utilizing eukaryotic organisms. These organisms generate energy in the form of adenosine triphosphate (ATP), and oxygen is reduced to water. Ninety percent of the oxygen uptake is consumed in mitochondria. A substantial byproduct of this ATP generation is the formation of potentially toxic oxygen radicals. Mitochondria damage results from elevated levels of ROS and is debilitating to eukaryotic cells. L-ergothioneine protects eukaryotic cellular mitochondria, U.S. Pat. No. 6,479,533, Yarosh, Nov. 12, 2002, incorporated herein by reference. Free radical scavenging improves survival of precursor and progenitor cells and improves secretory function in fully differentiated beta cell phenotypes.

Cell proliferation, cell growth, and differentiation of precursor and progenitor cells may be induced by biochemical agents consisting of growth factors, enzymes, hormones, peptides, and transcription factors added to cell culture media. Fibroblast growth factor (FGF) polypeptides are essential for, normal islet stem cell maintenance, proliferation, and generation of islets, U.S. Patent Application No. 20030138949, Bhushan, Anil, et al., Jul. 24, 2003, incorporated herein by reference. FGF signals pathway elements such as ligands, receptors and intracellular components to activate pancreatic stem cells and stimulate cell proliferation and cell growth. IGF-1 activates IGF-2 via mediated signal transduction which stimulates beta cell proliferation and growth, Lingohr, Melissa K. et al., Diabetes 51:966-976, 2002, incorporated herein by reference. The two growth factors may also induce differentiation in precursor and progenitor cell to beta cell phenotypes. In the presence of epidermal growth factor (EGF), the mass of tissue that develops is increased, yet, the absolute number of endocrine cells that develops is decreased, Cras-Méneur, C. et al., Diabetes 52:124-132, 2003, incorporated herein by reference. Under this condition, a large number of epithelial cells proliferate but remain undifferentiated. When EGF is removed from the immature pancreatic epithelial cells, the cells differentiate into insulin-expressing cells. Activating the proliferation of immature epithelial cells with EGF and then inducing their differentiation into endocrine cells by removing EGF increases the number of beta cell phenotypes. When basic-FGF (b-FGF) and EGF are added together to culture media containing islet cells, growth of NIP's is observed, Zulewski, H. et al., Diabetes 50:521-533, 2001. Hepatocyte growth factor (HGF) has surprisingly positive effects on beta cell mitogenesis, glucose sensing, and beta cell markers of differentiation, Garcia-Ocafia, A. et al., Diabetes 50:2752-2762, 2001, incorporated herein by reference. It has been postulated that HGF is an apoptosis inhibitor. HGF can also induce pancreatic islet cells to expand in vitro and/or in vivo, as do other factors including IGFs (IGF-1 and IGF-2), glucagon-like peptides (GLP-1 and GLP-2), prolactin (PRL), and exendins (EX-3 and EX-4), Tourrel C. et al., Diabetes 50:1562-1570, 2001. GLP-1 induces pancreatic beta cell proliferation and growth by increasing the expression level of the beta cell-specific pancreatic duodenal transcription factor (PDX-1), Buteau, J. et al., Diabetes 52:124-132, 2003, incorporated herein by reference. Transactivation of the EGF receptor, proteolytic processing of EGF-like ligands link GLP-1 receptor signaling to PI 3-kinase activation and beta cell proliferation. All-trans retinoic acid (AtRA) induces differentiation of ducts and endocrine cells, Tulachan, S. S. et al., Diabetes 52:76-84, 2003, incorporated herein by reference. AtRA has a wide spectrum of biological activities, including cell proliferation and growth, differentiation, and morphogenesis. AtRA upregulates PDX-1, an important transcription factor in pancreatic development. In the presence of exogenous AtRA, pancreatic progenitors differentiate into ducts and endocrine cells. PDX-1 is essential in the development and differentiation of pancreatic islets and in beta cell-specific gene expression, Marshak S. et al., Diabetes: 50, S131-S132, 2001. It hash extensive roles in regulating pancreas development and maintaining beta cell function and is a potent beta cell differentiation factor, Yoshida, S. et al., Diabetes 51:2505-2513, 2002, incorporated herein by reference. Nicotinamide (B3) also enhances cell proliferation of pancreatic stem cells in culture media and improves insulin secretion by beta cells, Ramiya, V. K. et al., Nature Medicine:6, 278-282, 2000. It is clear that growth factors enhance cell proliferation, cell growth, and even inhibits apoptosis. Adding biochemical agents consisting of growth factors, enzymes, hormones, peptides, vitamins, and transcription factors to cell culture media induces differentiation of precursors, pancreatic progenitor cells, and non-pancreatic progenitor cells.

Current liquid phase bioreactor technology and cell culture bioprocessing is lacking in advanced process control methodology of crucial cell culture process variables. Crucial cell culture process variables as well as their operating parameters have been neglected in liquid phase bioreactors. This compromises the cell culture process. Applying advanced process control methodology, cell culture process controls, and control of cell culture operating parameters can optimize mammalian cell culturing and cell proliferation, cell growth, and cellular differentiation.

Current liquid phase bioreactor technology incorporates methodologies to enhance the bioavailability of dissolved oxygen to cells in culture media. There are two major problems with these bioreactors, which include air lift bioreactors, membrane bioreactors, and two phase gas-liquid bioreactors. One problem with air lift and membrane bioreactors is that they only make oxygen bioavailable to cells in culture media at the solubility limit of oxygen in water, approximately 6.83 milligrams of oxygen per liter of water at 37° C. Diffusion limitations in the culture media limit the bioavailability of oxygen to cells in these liquid bioreactors. Another problem, encountered with two phase gas-liquid bioreactors, is caused by the gas-liquid interfaces at the surfaces of solid cells in the aqueous media and gas bubbles the cells are in intimate contact with. Hydrodynamic and shear forces are very high at the gas-liquid-solid interface at the cell's outer surface, and animal and mammalian cells are broken and damaged by shear force as they stretch over the surface of bubbles.

In U.S. Pat. No. 6,664,095, Suryanarayan, et al., Dec. 16, 2003 incorporated herein by reference, bioreactor control is applied only to solid state fermentation utilizing solid state media. It is not applied to a liquid bioreactor utilizing liquid culture media to proliferate, expand, and grow cells. It is limited to temperature control but the means and methodology of process control are neglected. There is no application of advanced process control methodology and no control of the process variables via process control apparatus; there is no control hardware, no microprocessor control, no crucial variable process sensors, no control setpoints, and no control (setpoint) parameters. This is certainly an inefficient method of bioreactor operation and in need of improvement.

In U.S. Pat. No. 6,455,306, Goldstein, et al., Sep. 24, 2002, incorporated herein by reference, the culture media contains perfluorocarbon to improve the exchange of oxygen between cells and the culture media. This is accomplished without the addition of a continuous phase stabilizing surfactant. Creaming and coalescence of the perfluorocarbon phase occur without a continuous phase stabilizing surfactant. This is a certainly an inefficient method to increase bioavailable oxygen and in need of improvement.

In U.S. Pat. No. 6,156,570; Hu, et al., Dec. 5, 2000, incorporated herein by reference, there is no mention of bioreactor control at all. Oxygen consumption is measured and a ratio of glucose to oxygen is calculated, but this is to maintain the glucose level and not control the oxygen concentration in the culture media. There is no application of advanced process control methodology, no control hardware, no microprocessor control, no crucial variable process sensors, no control setpoints, and no control (setpoint) parameters. This is certainly an inefficient method of bioreactor operation and in need of improvement.

In U.S. Pat. No. 5,637,496, Thaler et al., Jun. 10, 1997, incorporated herein by reference, there is again no mention of bioreactor control at all. A temperature sensor is mentioned, but the means and methodology of process control are neglected. There is no application of advanced process control methodology and no temperature control via process control apparatus. There is no process control hardware, no microprocessor control of other crucial process variables, no control setpoints, and no control (setpoint) parameters. This is certainly an inefficient method of bioreactor operation and in need of improvement.

In U.S. Pat. No. 5,629,202, Su, et al., May 13, 1997, a computer is used to control feeding pumps which feed indole, or pyruvic acid, or pyruvate salt, or ammonium salt into the bioreactor to optimize tryptophanse's activity to produce L-tryptophan in the bioreactor. There is no application of advanced process control methodology, no control hardware, no microprocessor control of other crucial process variables, no process sensors, no control setpoints, and no control (setpoint) parameters. This is certainly an inefficient method of bioreactor operation and in need of improvement.

In U.S. Patent Application No. 20030199083, Vilendrer, Kent; et al., Oct. 23, 2003, incorporated herein by reference, a microprocessor is used to control the fluid flow while conditioning intravascular tissue products for bioprothesis. Oxygen, carbon dioxide, and temperature control is only specified with respect to conditioning intravascular tissue products consisting of endothelial, smooth muscle, and fibroblast cells for bioprothesis. There are no control setpoints, and no control (setpoint) parameters. Microprocessor control is not specified nor claimed with respect to culturing, proliferation, and growth of non-differentiated precursor and progenitor cells in bioreactors. The method of intravascular tissue conditioning is not the method of precursor and progenitor cell culturing, precursor and progenitor cell proliferation and growth, and inducing differentiation in non-differentiated precursor and progenitor cells.

In New Zealand Patent 509,669, Benedict, Daniel, February 2004, incorporated herein by reference, microprocessor control is used to control process variables at setpoints while isolating fully differentiated islet cells from a pancreas. An automated method of islet isolation is specified and claimed. Microprocessor control is not specified nor claimed with respect to culturing and growing non-differentiated precursor and progenitor cells in a bioreactor. Microprocessor control in not specified nor claimed with respect to inducing differentiation in precursor and progenitor cells in two liquid phase bioreactors. A method of isolating fully differentiated islet cells from pancreata is not similar to a method of precursor and progenitor cell culturing, precursor and progenitor cell proliferation and growth, and inducing differentiation in non-differentiated precursor and progenitor cells in two liquid phase bioreactors.

Current cell culturing methods in a liquid bioreactor do not recognize or control all the crucial cell culture process variables of the cell culture media that may be controlled to optimize animal and mammalian cell proliferation and growth. This can be improved.

Current cell culturing methods in a liquid bioreactor do not recognize or add all the growth factors, or enzymes, or hormones, or peptides, or transcription factors that may be added to cell culture media to optimize cell proliferation and growth. This can be improved.

Current cell culturing methods in a liquid bioreactor do not identify or add all the growth factors, or enzymes, or hormones, or peptides, or transcription factors that may be added to cell culture media to induce cellular differentiation of ES cells, endocrine progenitor cells, pancreatic progenitor cells, pancreatic stem cells, pancreatic duct epithelial cells, nestin-positive islet-derived progenitor cells (NIPs), or pluripotent non-embryonic stem (PNES) cells in a bioreactor. This can be improved.

Current cell culturing methods in a liquid bioreactor do not present the greatest concentrations of bioavailable oxygen or carbon dioxide to cells in liquid cell culture media. This can be improved in two liquid phase bioreactors where the second liquid phase is organic.

### BRIEF SUMMARY OF THE INVENTION

It is the object of this invention to present Advanced Bioreactor Cell Culture Technology, a method of advanced animal and mammalian cell culturing and bioprocessing incorporating molecular biology techniques, advanced process control methodology, and a process control interface applied to cell culturing and liquid phase bioprocessing of animal and mammalian cells in two liquid phase bioreactors.

The present invention relates to the application of molecular biology techniques to animal and mammalian cell culturing and the addition of specific biochemical agents consisting of growth factors, enzymes, hormones, peptides, and transcription factors to two liquid phase cell culture bioreactor to optimize animal and mammalian cell culturing and maximize cell proliferation and growth of but not limited to, precursor cells, ES cells, endocrine progenitor cells, pancreatic progenitor cells, pancreatic stem cells, pancreatic duct epithelial cells, nestin-positive islet-derived progenitor cells (NIPs), or pluripotent non-embryonic stem (PNES) cells in a bioreactor. Biochemical agents consisting of growth factors, hormones, and peptides such as EGF, EX-3, EX-4, b-FGF (basic), GLP-1, GLP-2, HGF, IGF-1, IGF-2, and prolactin added to cell culture media stimulate proliferation and growth of precursor and progenitor cells. Addition of growth factors, hormones, and peptides to cell culture media improves cell proliferation and growth during cell culturing in a liquid bioreactor.

The present invention also relates to the application of molecular biology techniques to animal and mammalian cell culturing and the addition of specific biochemical agents consisting of growth factors and transcription factors to a liquid phase cell culture bioreactor to influence, stimulate, and induce differentiation of but not limited to, precursor cells, ES cells, endocrine progenitor cells, pancreatic progenitor cells, pancreatic stem cells, pancreatic duct epithelial cells, nestin-positive islet-derived progenitor cells (NIPs), or pluripotent non-embryonic stem (PNES) cells in a bioreactor to beta cell phenotypes. Biochemical agents consisting of transcription, growth factors, and vitamins; IGF-1, IGF-2, PDX-1, AtRA, B3 added to cell culture media influences, stimulates, and induces differentiation of precursor and progenitor cells; while the removal of EGR from cell culture media influences, stimulates, and induces cellular differentiation. Addition of transcription and growth factors to cell culture media will improve differentiation during cell culturing in a liquid bioreactor.

The present invention further relates to the application molecular techniques biology to animal and mammalian cell culturing and the addition of specific biochemical agents consisting of growth factors, enzymes, amino acids, and antibiotics to a liquid phase cell culture bioreactor to inhibit nitric oxide formation and apoptosis in animal and mammalian cell culturing. Biochemical agent consisting of derivatives of L-arginine, diaminoarginine, methlyarginine, dimethylarginine, and 2,4-diamino-6-hydroxypyrmidine, tetracycline, minocycline, doxycycline, cysteine, cystine, dextran, heparin, IGF-1, and IGF-2 added to the culture media may inhibit nitric oxide formation and subsequent apoptosis; while radical scavenging of ROS by SODs, Se-SOD. Mn-SOD, Zn-SOD, ZnCu-SOD, GR, GSHpx, and L-ergothioneine added to the culture media may inhibit apoptosis. Endotoxin neutralizing protein (ENP) added to the culture media also inhibits apoptosis by neutralizing endotoxin in the culture media. Addition of ROS scavengers, growth factors, enzymes, amino acids, and antibiotics will improve cell culturing in a two liquid phase bioreactor.

The present invention still further relates to the application of advanced process control methodology and a process control interface to a liquid phase cell culture bioreactor to optimize animal and mammalian cell culturing, and maximize cell proliferation and growth of but not limited to, precursor cells, ES cells, endocrine progenitor cells, pancreatic progenitor cells, pancreatic stem cells, pancreatic duct epithelial cells, nestin-positive islet-derived progenitor cells (NIPs), or pluripotent non-embryonic stem (PNES) cells in a bioreactor by controlling crucial cell culture process variables. Important and crucial cell culture process variables include the cell culture media temperature (T), the cell culture media hydrogen (pH) ion concentration, the cell culture media dissolved oxygen (DO) concentration, the cell culture media dissolved carbon dioxide (CO₂) concentration, the cell culture media nitric oxide (NO) concentration, the cell culture media endotoxin (E) concentration, the cell culture media antibiotic (A) concentration, and the cell culture media agitator (S) speed. Control of these crucial cell culture process variables will improve cell culturing in a liquid bioreactor.

The present invention employs advanced process control methodology, a process control interface, and process control hardware to automate cell culturing in a liquid phase bioreactor. Microprocessor controllers, process sensors, control setpoints, and control (setpoint) parameters are used to control crucial cell culture process variables. Control of the crucial process variables that influence cell culturing in a liquid bioreactor are incorporated into the method of cell culturing by integrating automated control of the cell culturing process variables via microprocessor controllers through the process control interface to the liquid bioreactor, utilizing an analog and digital (A/D) electrical (electronic) interface, with feedback from process sensors in the bioreactor to control cell culturing. Data acquisition (DAQ) during cell culturing is accomplished with a windows based computer (PC) operating in the (LabView) graphical software-programming environment (G). Analog and digital output from the DAQ PC via the analog-digital (A/D) interface is used to activate electric solenoid valves controlling bioreactor perfusion with culture media, recirculation of the liquid phases, and removal of spent culture media. Cell culture data are displayed in real-time on an interactive graphical process flowsheet displaying cell culture and bioreactor status. Pumps, valves, thermocouples, sensors, and probes are located on the graphical process flowsheet (computer display) and correspond to their actual physical location on the bioreactor. Cell culture process data are acquired during cell culturing from the microprocessor controllers and process sensors located in the bioreactor and logged to a data file for post-processing analysis, quality assurance, validation, and regulatory purposes.

The present invention presents the design of novel two liquid phase bioreactors that incorporates a second liquid organic phase to increase the bioavailability of oxygen to cells in aqueous phase culture media in the bioreactor and to control the pH of the culture media. Both phases are removed from the bioreactor thru a single exit for phase separation and recycle. The two phase liquid recycle first enters a phase separator (settling chamber) and the aqueous phase with culture media is separated and directly recycled as a single phase to the bioreactor without enrichment in oxygen or carbon dioxide, via a separate inlet (aqueous phase only) to the bioreactor. The second liquid phase, now a single organic phase without culture media, is further conveyed to a gas phase contactor (bubble chamber) and enriched with oxygen and carbon dioxide, and then recycled as a single enriched organic phase to the bioreactor. The second organic liquid phase is added via a separate inlet (organic phase only) to the bioreactor in which the non-emulsified organic compound enriched outside of the bioreactor is mixed with the aqueous phase in the bioreactor, making high concentrations of oxygen bioavailable to cells in the culture media and also controlling the culture media pH. The second liquid phase is immiscible in water. The second liquid phase can be a perfluorocarbon compound, say 1-bromoheptadecafluorooctane or octadecafluorooctane, which has solubility limits for oxygen and carbon dioxide many times greater than that of water, or a long chain aliphatic carbon compound, say tetradecane or dodecane, which has solubility limits for oxygen and carbon dioxide at least eight times that of water. This greatly increases the bioavailability of oxygen to cells in the aqueous phase inside the bioreactor and facilitates pH control of the culture media. Addition of a continuous phase stabilizing surfactant to the aqueous phase inhibits creaming and coalescence of the organic phase. Diffusion limitations of oxygen transfer to cells in culture media in airlift and membrane bioreactors are overcome. Another function of a two liquid phase bioreactor is that it eliminates the high shear forces present at cell gas-liquid-solid interfaces of cells in contact with bubbles in liquid culture media. This prevents cellular damage caused by hydrodynamic and shear forces when cells rupture cells as they stretch over bubble surfaces. Increasing the bioavailability of oxygen, and preventing cellular damage due to hydrodynamic and shear forces enhances cell culturing in a liquid bioreactor. Increasing the bioavailability of oxygen to cells in liquid culture media, eliminating shear forces at cell surfaces, and facilitating pH control of the culture media will improve cell culturing in a liquid bioreactor.

This invention provides a method that is superior to the current and inefficient methods of animal and mammalian cell culturing through application of molecular biology techniques, by addition of specific biochemical agents consisting of growth factors, peptides, enzymes, vitamins, and transcription factors to liquid phase cell culture media to optimize and maximize cell proliferation and growth.

This invention also provides a method that is superior to the current and inefficient methods of animal and mammalian cell culturing through application of molecular biology techniques, by addition of specific biochemical agents consisting of growth factors and transcription factors to liquid phase cell culture media to influence, encourage, and induce differentiation of precursor and progenitor cells to beta cell phenotypes.

This invention also provides a method that is superior to the current and inefficient methods of animal and mammalian cell culturing through application of molecular biology techniques, by addition of specific biochemical agents consisting of free radical scavenging enzymes, hormones, amino acids, antibiotics, and endotoxin neutralizing protein to liquid phase cell media to inhibit nitric oxide formation and apoptosis in animal and mammalian cell culturing.

This invention further provides a method that is superior to the current and inefficient methods of bioreactor operation, by applying advanced process control methodology to the important and crucial cell culture process variables, and controlling the crucial cell culture process variables that influence and control cellular proliferation and growth dynamics during bioprocessing and cell culturing.

This invention further provides a bioreactor design and method of oxygenation and pH control that is superior to the current methods of delivering bioavailable oxygen to cells in liquid culture media, by increasing the bioavailability of oxygen and carbon dioxide to cell culture media, by overcoming the hydrodynamic and diffusion limitations of current bioreactors, through design of novel two liquid phase bioreactors for cell culturing and bioprocessing.

Advanced Bioreactor Cell Culture Technology applies advanced molecular biology techniques to animal and mammalian cell culturing by addition of specific biochemical agents consisting of growth factors, enzymes, peptides, transcription factors, hormones, amino acids, vitamins, and antibiotics to animal and mammalian cell culturing media; increases the bioavailability of oxygen to liquid cell culture media; and applies advanced process control methodology which involves complete process control and includes microprocessor controllers, process sensors, control setpoints, and control (setpoint) parameters to control crucial cell culture process variables.

The above is a brief description of the advantages of the present invention. The features, embodiments, and advantages of the invention will be apparent to those skilled in the science from the accompanying drawings, following description, and appended claims.

### BRIEF DESCRIPTION OF THE SEVERAL VIEWS OF THE DRAWINGS

FIG. 1 is a process flowsheet of a two liquid phase cell culture bioreactor showing the recirculation paths of the aqueous and organic liquid phases, interconnection of the process components, microprocessor controllers, process sensors, analog and digital electrical (electronic) process control interface, and data acquisition computer according to one preferred embodiment of the invention.

FIG. 2 is a process flowsheet of a two liquid phase cell culture bioreactor showing the recirculation paths of the aqueous and organic phases, the interconnection of the process components, microprocessor controllers, process sensors, analog and digital electrical (electronic) process control interface, and data acquisition computer according to one preferred embodiment of the invention.

FIG. 3 is a schematic and block diagram of a two liquid phase bioreactor showing the interconnection of the process components, microprocessor controllers, process sensors, process valves, analog and digital electrical (electronic) process control interface, and data acquisition computer according to one preferred embodiment of the invention.

### DETAILED DESCRIPTION OF THE INVENTION

The invention is directed at an improved method of culturing, proliferating, growing, and inducing differentiation in animal and mammalian precursor cells, ES cells, endocrine progenitor cells, pancreatic progenitor cells, pancreatic stem cells, pancreatic duct epithelial cells, nestin-positive islet-derived progenitor cells (NIPs), or pluripotent stem cells, or pluripotent non-embryonic stem (PNES) cells in a bioreactor through application of molecular biology techniques. The invention is also directed at an improved method of culturing, proliferating, growing, and inducing differentiation in animal or mammalian precursor cells by application of advanced process control methodology, microprocessor controllers, process sensors, control setpoints, and control (setpoint) parameters are used to control crucial cell culture process during cell culturing.

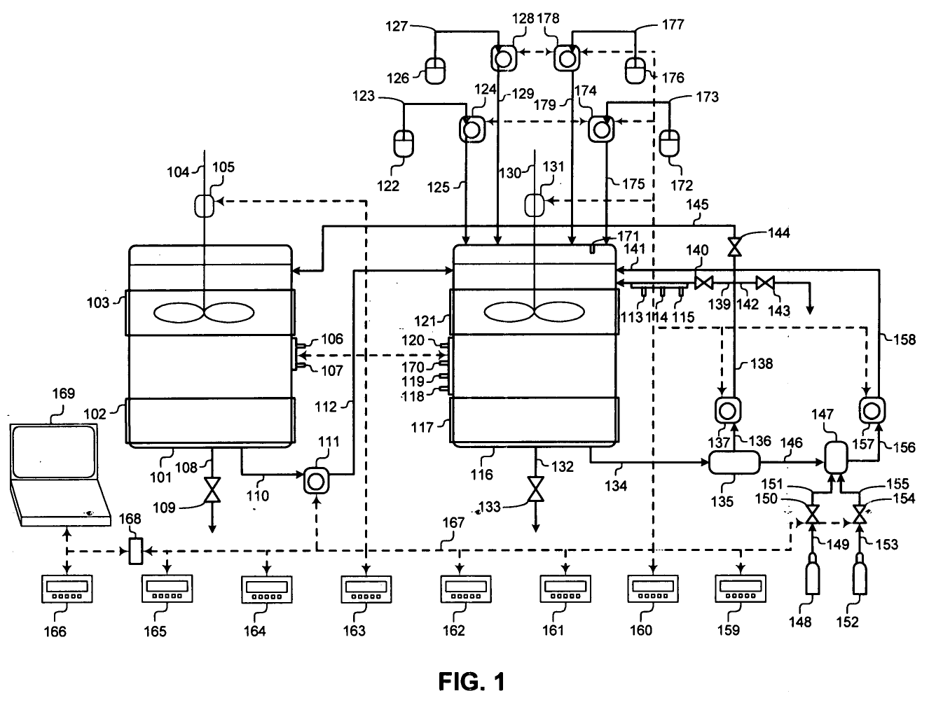

FIG. 1 illustrates a process flowsheet demonstrating the interworking of the various components of a two phase liquid bioreactor according to one preferred embodiment of the invention. As shown in FIG. 1, fresh and sterile culture media is temperature controlled in media reservoir 101 by process heaters 102 and 103 and kept in suspension by impeller 104 and stirring motor 105. Culture media may be removed from the media reservoir via drain line 108 and drain valve 109. The temperature in the media reservoir is monitored by temperature sensor 106 and the pH is monitored by pH sensor 107. The culture media is pumped from the reservoir through process line 110 by feed pump 111 and through process line 112 into the two liquid phase bioreactor 116, which is temperature controlled by process heaters 117 and 121. Culture media and cells may be removed from the bioreactor via drain line 132 and drain valve 133. The temperature of the culture media in the bioreactor is controlled by temperature sensor 120 and temperature controller 166 and kept in suspension by impeller 130 and stirring motor 131. The pH of the culture media is controlled by pH sensor 119 and pH controller 165 and by addition of base from base reservoir 122 through process line 123 and base pump 124 through base feed line 125 to the bioreactor. The pH of the culture media is controlled by the pH 119 sensor and pH controller 165 by addition of acid from acid reservoir 126 through process line 127 and acid pump 128 through acid feed line 129 to the bioreactor. The endotoxin concentration in the culture media is controlled by endotoxin sensor 118 and endotoxin neutralizing protein controller 161 by addition of endotoxin neutralizing protein from endotoxin neutralizing protein reservoir 176 thru process line 177 and endotoxin neutralizing protein pump 178 through endotoxin feed line 178. Nitric oxide in the bioreactor is monitored by nitric oxide sensor 170 and nitric oxide meter 159. The two liquid phases, aqueous and organic, are circulated thru process line 134 to phase separator (settling tank) 136 and the aqueous phase is recycled to the bioreactor through process line 136 by recycle pump 137 through process line 139 through process valve 140. The dissolved oxygen concentration of the aqueous phase is controlled by dissolved oxygen probe 113 and dissolved oxygen controller 164 and oxygen is added to the organic phase from oxygen tank 148 through process line 149 through oxygen valve 150 through process line 151 to the phase contactor (bubble chamber) 147. The dissolved carbon dioxide concentration of the aqueous phase is controlled by carbon dioxide probe 114 and carbon dioxide controller 163 and carbon dioxide is added to the organic phase from carbon dioxide tank 152 through process line 153 through carbon dioxide valve 154 through process line 155 to the phase contactor. After separation in the phase separator, the organic phase is recycled to the bioreactor through process line 146 to the phase contactor for oxygen and carbon dioxide enrichment and recycled to the bioreactor through process line 156 by recycle pump 157 through process line 158. The antibiotic concentration is controlled by antibiotic sensor 115 and by antibiotic controller 162 by addition of antibiotic from antibiotic 172 thru process line 173 and antibiotic pump 174 through antibiotic feed line 175. Alternatively, the aqueous phase may be recycled to the media reservoir through the phase separator by the recycle pump through process valve 144 through process line 145. During perfusion and addition of fresh culture media to the bioreactor, spent media flows through the phase separator by the recycle pump and is removed from the bioreactor through drain line 142 through drain valve 143. The liquid level in the bioreactor is controlled by liquid level sensor 171 and liquid level controller 160.

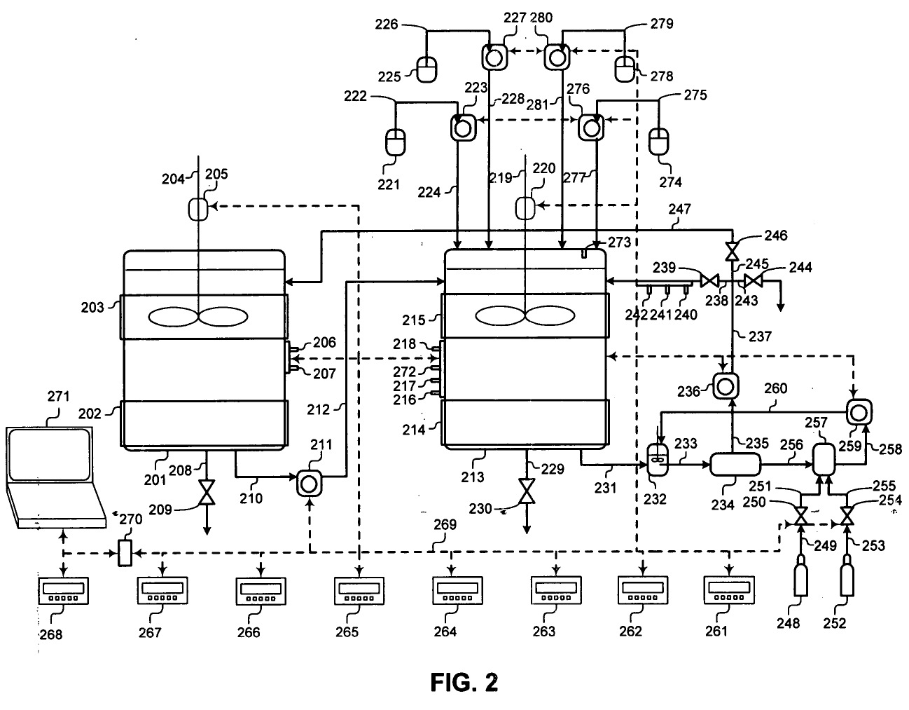

FIG. 2 illustrates a process flowsheet demonstrating the interworking of the various components of a two phase liquid bioreactor according to one preferred embodiment of the invention. As shown in FIG. 2, fresh and sterile culture media is temperature controlled in media reservoir 201 by process heaters 202 and 203 and kept in suspension by impeller 204 and stirring motor 205. Culture media may be removed from the media reservoir via drain line 208 and drain valve 209. The temperature in the media reservoir is monitored by temperature sensor 206 and the pH is monitored by pH sensor 207. The culture media is pumped from the reservoir through process line 221 by feed pump 211 and through process line 212 into the two liquid phase bioreactor 213, which is temperature controlled by process heaters 214 and 215. Culture media and cells may be removed from the bioreactor via drain line 229 and drain valve 230. The temperature of the culture media in the bioreactor is controlled by temperature sensor 218 and temperature controller 266 and kept in suspension by impeller 219 and stirring motor 220. The pH of the culture media is controlled by pH sensor 216 and pH controller 265 and by addition of base from base reservoir 221 through process line 222 and base pump 223 through base feed line 224 to the bioreactor. The pH of the culture media is also controlled by the pH sensor and the pH controller by addition of acid from acid reservoir 226 through process line 227 and acid pump 228 through acid feed line 229 to the bioreactor. The endotoxin concentration in the culture media is controlled by endotoxin sensor 217 and endotoxin neutralizing protein controller 261 by addition of endotoxin neutralizing protein from endotoxin neutralizing protein reservoir 278 thru process line 279 and endotoxin neutralizing protein pump 280 through endotoxin feed line 281. Nitric oxide in the bioreactor is monitored by nitric oxide sensor 272 and nitric oxide meter 259. The aqueous phases is circulated thru process line 231 to the liquid phase contactor 232 and the two phases exit the phase contactor through process line 233 to the phase separator (settling tank) 234. The enriched aqueous phase is recycled to the bioreactor through process line 235 by recycle pump 236 through process line 237 through process valve 246 through process line 247. The dissolved oxygen concentration of the aqueous phase is controlled by dissolved oxygen probe 242 and dissolved oxygen controller 164, and oxygen is added to the organic phase from oxygen tank 248 through process line 249 through oxygen valve 250 through process line 251 to the phase contactor (bubble chamber) 247. The dissolved carbon dioxide concentration of the aqueous phase is controlled by carbon dioxide probe 241 and carbon dioxide controller 263 and carbon dioxide is added to the organic phase from carbon dioxide tank 252 through process line 253 through carbon dioxide valve 254 through process line 255 to the phase contactor. After separation in the phase separator, the organic phase exits the phase separator through process line 256 to the phase contactor (bubble chamber) 257. The organic phase is recycled to the liquid phase contactor through process line 258 through recycle pump 259 through process line 260 for oxygen and carbon dioxide enrichment of the aqueous phase. The antibiotic concentration is controlled by antibiotic sensor 240 and by antibiotic controller 262 by addition of antibiotic from antibiotic 274 thru process line 275 and antibiotic pump 276 through antibiotic feed line 277. Alternatively, the aqueous phase may be recycled to the media reservoir through the phase separator by the recycle pump through process valve 245 through process line 246. During perfusion and addition of fresh culture media to the bioreactor, spent media flows through the phase separator by the recycle pump and is removed from the bioreactor through drain line 243 through drain valve 244. The liquid level in the bioreactor is controlled by liquid level sensor 273 and liquid level controller 260.

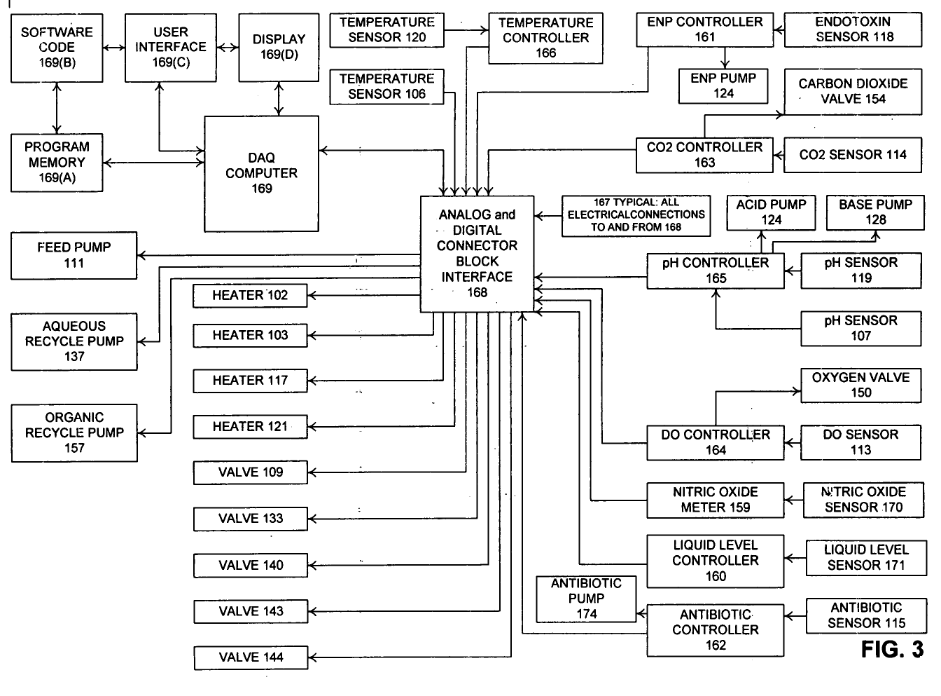

FIG. 3 illustrates a schematic and block diagram of the interaction of the various components of the two liquid phase bioreactor, electrical connections and wiring according to one preferred embodiment of the invention. Electrical process connections 167 interface the process sensors 106, 107, 113, 114, 115, 118, 119, 120, 170, 171, previously described in FIG. 1, the microprocessor controllers 160, 161, 162, 163, 164, 165, 166, previously described in FIG. 1, and the microprocessor meter 160, previously described in FIG. 1, through the analog and digital connector block interface 168 to the liquid bioreactor. A computer is used for data acquisition (DAQ) 169 and is also employed to control the electric solenoid process valves 109, 133, 140, 143, 144 and gas valves 150 and 154, previously described in FIG. 1, to record the processing data via real-time DAQ, consisting of output from microprocessor controllers, microprocessor meter, process sensors, through electrical (electronic) process connections 167 and the analog and digital and connector block interface 168. The microprocessor computer 169 consists of the program memory 169(A), random access memory (RAM) and read only memory (ROM), stored by a hard-drive (HD) and or erasable programmable read only memory (EPROM), software code 169(B), stored by either RAM, ROM, EPROM, or HD, and user interface 169(C) incorporating keyboard, mouse, interconnection cables and a numerical and graphical display (computer monitor) 169(D).

The advantages of the present invention utilizing molecular biology techniques and advanced process control methodology, may be also applied to single liquid phase cell culture bioreactors, including air lift bioreactors, membrane bioreactors, and gas-liquid bioreactors.

All publications, patents, and patent documents are incorporated herein by reference, as though individually incorporated by reference. The invention has been described with reference to various specific and preferred embodiments and techniques. However, it should be understood that many variations and modifications might be made while remaining within the spirit and scope of the invention. The above descriptions of exemplary embodiments are for illustrative purposes. Because of variations that will be apparent to those skilled in the science, the present invention is not intended to be limited to the particular embodiments described above. Thus, various modifications of the above-described embodiments will be apparent to those skilled in the art or science. The present invention may also be practiced in the absence of any element not specifically disclosed. The invention may be uniformly applied to animal and mammalian cell culturing in a two-liquid phase bioreactor with cells derived from animals and mammals either transgenic or non-transgenic. The scope of the invention is defined by the following claims.

1. A method of culturing cells in a two liquid phase bioreactor wherein:
(a) a biochemical agent is added to a liquid culture media to induce precursor or progenitor cells to proliferate, or grow, or expand their number without substantial differentiation;
(b) the cells are embryonic stem cells (ES), or endocrine progenitor cells, or pancreatic progenitor cells, or pancreatic stem cells, or pancreatic duct epithelial cells, or nestin-positive islet-derived progenitor cells (NIP)s, or pluripotent stem cells, or pluripotent non-embryonic stem (PNES);
(c) the biochemical agent is a growth factor or a hormone;
(d) the growth factor is IGF-1 (Insulin like Growth Factor-1), or IGF-2 (Insulin like Growth Factor-2), or b-FGF (basic-Fibroblast Growth Factor), or EGF (epithelial growth factor), or HGF (Hepatocyte Growth Factor), or any combination thereof;
(e) the hormone is PRL (prolactin);
(e) the cells are human cells, or transgenic mammalian cells, or non-transgenic mammalian cells, or transgenic porcine cells, or non-transgenic porcine cells, or transgenic animal cells, or non-transgenic animal cells, or transgenic fish cells.

2. The method of claim one wherein:
(a) a second biochemical agent is added to a liquid culture media to induce precursor or progenitor cells to proliferate, or grow, or expand their number without substantial differentiation;
(b) the second biochemical agent is the peptide EX-3 (exendin-3), or EX-4 (exendin-4), or GLP-1 (glucagon like peptide-1), or GLP-2 (glucagon like peptide-2), or any combination thereof.

3. The method of claim one wherein:
(a) a biochemical agent is added to a liquid culture media to control or inhibit nitric oxide (NO) formation or inhibit apoptosis;
(b) the biochemical agent is a growth factor;
(c) the growth factor is IGF-1 (Insulin like Growth Factor-1), or IGF-2 (Insulin like Growth Factor-2), or HGH (Hepatocyte Growth Factor), or any combination thereof.

4. The method of claim one wherein:
(a) a biochemical agent is added to a liquid culture media to control or inhibit nitric oxide (NO) formation or inhibit apoptosis;
(b) the biochemical agent is an antibiotic;
(c) the antibiotic is tetracycline, or doxycycline, or minocycline, or any combination thereof.

5. The method of claim one wherein:
(a) a biochemical agent is added to a liquid culture media to control or inhibit nitric oxide (NO) formation or inhibit apoptosis;
(b) the biochemical agent is an anticoagulant;
(c) the anticoagulant is heparin.

6. The method of claim one wherein:
(a) a biochemical agent is added to a liquid culture media to control or inhibit oxide (NO) formation or inhibit apoptosis;
(b) the biochemical agent is an nitrogenous base;
(c) the biochemical is N.N-diaminoguanidine, or methylguanidine, or 1,1-dimethylguanadine, or 2,4-diamina-6-hydroxypryrmidine, or any combination thereof.

7. The method of claim one wherein:
(a) a biochemical agent is added to a liquid culture media to control or inhibit oxide (NO) formation or inhibit apoptosis;
(b) the biochemical agent is an amino acid;
(c) the amino acid is cysteine, or cystine, or any combination thereof.

8. The method of claim one wherein:
(a) a biochemical agent is added to a liquid culture media to control or inhibit oxide (NO) formation or inhibit apoptosis;
(b) the biochemical agent is polysaccharide;
(c) the polysaccharide is dextran.

9. The method of claim one wherein:
(a) a biochemical agent is added to a liquid culture media to control or inhibit nitric oxide synthase (NOS) activity or inhibit apoptosis;
(b) the biochemical agent is a growth factor;
(c) the growth factor is IGL-1 (Insulin like Growth Factor-1).

10. The method of claim one wherein:
(a) a biochemical agent is added to a liquid culture media to control or scavenge reactive oxide species (ROS) or inhibit apoptosis;
(b) the biochemical agent is an enzyme;
(c) the enzyme is SOD (super oxide dismutase), or Se-SOD (Selenium dependent super oxide dismutase, or Mn-SOD (magnesium super oxide dismutase), or Zn-SOD (zinc super oxide dismutase, or ZnCu-SOD (zinc copper superoxide dismutase), or GSHpx (glutathione peroxidase), or GR (glutathione reductase), or catalase, or any combination thereof.

11. The method of claim one wherein:
(a) a biochemical agent is added to a liquid culture media to control or scavenge reactive oxide species (ROS) or inhibit apoptosis;
(b) the biochemical agent is an antioxidant in the chemical class: 2-thio-imidazole, amino acid;
(c) the chemical agent is L-ergothioneine.

12. The method of claim one wherein:
(a) a biochemical agent is added to a liquid culture media to induce precursor or progenitor cells to differentiate into beta cell phenotypes without substantial proliferation;
(b) the biochemical agent is a growth factor;
(c) the growth factor is IGF-1 (Insulin like Growth Factor-1), or IGF-2 (Insulin like Growth Factor-2), or any combination thereof.

13. The method of claim one wherein:
(a) a biochemical agent is added to a liquid culture media to induce precursor or progenitor cells to differentiate into beta cell phenotypes without substantial proliferation;
(b) the biochemical agent is a vitamin;
(c) the vitamin is B3 (Nicotinamide).

14. The method of claim one wherein:
(a) a biochemical agent is added to a liquid culture media to induce precursor or progenitor cells to differentiate into beta cell phenotypes without substantial proliferation;
(b) the biochemical agent is a transcription factor;
(c) the transcription factor is PDX-1 (Pancreatic Duodenal transcription factor).

15. The method of claim one wherein:
(a) a biochemical agent is added to a liquid culture media to induce precursor or progenitor cells to differentiate into beta cell phenotypes without substantial proliferation;
(b) the biochemical agent is a carboxylic acid;
(c) the carboxylic acid is all-trans retinoic acid.

16. The method of claim one wherein:
(a) a biochemical agent is removed from a liquid culture media to induce precursor or progenitor cells to differentiate into beta cell phenotypes without substantial proliferation;
(b) the biochemical agent is a growth factor;
(c) the growth factor is EGF (epithelial growth factor).

17. A method of culturing cells in a two liquid phase bioreactor wherein:
(a) a process variable describing the chemical character of the liquid, cell culture media is the process temperature (T);
(b) the process variable of the liquid cell culture media is directly controlled with a process controller via a setpoint;
(c) the setpoint is between 4.0 degrees Celsius and 44.0 degrees Celsius;
(d) the cells are human cells, or transgenic mammalian cells, or non-transgenic mammalian cells, or transgenic porcine cells, or non-transgenic porcine cells, or transgenic animal cells, or non-transgenic animal cells, or transgenic fish cells.

18. The method of claim 17 wherein the process controller is a PID (proportional, integral, derivative) controller.

19. The method of claim 17 wherein the process controller is a microprocessor temperature controller.

20. The method of claim 17 wherein the process controller is a microprocessor controller.

21. The method of claim 17 wherein the process controller is a variable resistance transformer.

22. The method of claim 17 wherein the process temperature is generated by an electrical resistance element.

23. The method of claim 17 wherein the process temperature is generated by steam.

24. The method of claim 17 wherein the process temperature is generated by a recirculating fluid bath.

25. The method of claim 17 wherein a second process variable is controlled:
(a) a second process variable is the process hydrogen ion concentration (pH);
(b) a second process controller is a microprocessor (pH) controller;
(c) the process pH setpoint is between pH 6.00 and pH 8.00.

26. The method of claim 17 wherein:
(a) a second process variable is the process hydrogen concentration (pH);
(b) the process pH is controlled by the addition of an acid or base to the cell culture media thereby adjusting or controlling the pH;
(c) the process pH is between pH 6.00 and pH 8.00.

27. The method of claim 17 wherein a second process variable is controlled:
(a) a second process variable is the process dissolved oxygen (DO) concentration;
(b) a second process controller is a microprocessor (DO) controller;
(c) the process DO concentration setpoint is between 0.000000001 milligrams per milliliter 0.000000001 mg/ml) DO and 2.0 milligrams per milliliter (2.0 mg/ml) DO.

28. The method of claim 17 wherein a second process control variable is controlled:
(a) a second process variable is the process dissolved nitric oxide (NO) concentration;
(b) a second process controller is a microprocessor NO controller;
(c) the process dissolved NO concentration setpoint is between 0.00000000000001 moles per liter (0.01 picomoles/liter) NO and 0.1 mole per liter (0.1 mol/liter) NO.

29. The method of claim 17 wherein a second process control variable is controlled:
(a) a second process variable is the process endotoxin (E) concentration;
(b) a second process controller is a microprocessor (E) controller;
(c) the process E concentration setpoint is between 0.000000001 endotoxin units (EU) per milligram (1.0 nanoEU/mg) and 100.0 endotoxin units per milligram (100.0 EU/mg).

30. The method of claim 17 wherein a second process control variable is controlled:
(a) a second process variable is the process endotoxin (E) concentration;
(b) a process endotoxin concentration is controlled by the addition of endotoxin neutralizing protein (ENP) to the process solution thereby neutralizing endotoxin in the process solution.

31. The method of claim 17 wherein a second process control variable is controlled:
(a) a second process variable is the process endotoxin neutralizing protein (ENP) concentration;
(b) a second process controller is a microprocessor ENP controller;
(c) the process ENP concentration setpoint is between 0.00000000000001 moles per liter (0.01 picomoles/liter) ENP and 0.1 moles per liter (0.1 mol/liter) ENP.

32. The method of claim 17 wherein a second process control variable is controlled:
(a) a second process variable is the process antibiotic (A) concentration;
(b) a second process controller is a microprocessor A controller;
(c) the process A concentration setpoint is between 0.00000000000001 moles per liter (0.01 picomoles/liter) A and 0.1 mole per liter (0.1 mol/liter) A.

33. The method of claim 17 wherein cell culturing proceeds automatically through a process control interface.

34. An apparatus for culturing cells comprising:
(a) a two liquid phase bioreactor;
(b) a culture media reservoir;
(c) a phase separator or setting tank;
(d) a phase contactor or bubble chamber;
(e) two distinct immiscible liquid phases wherein one phase is aqueous and the other phase is organic;
(f) both phases added separately to the bioreactor via separate inlets wherein each phase is not emulsified;
(g) a continuous phase stabilizing surfactant added to the aqueous phase;
(h) a second organic phase utilized to increase the bioavailability of oxygen to cells in the aqueous phase;
(i) both phases exit the bioreactor simultaneously via a single exit stream wherein both phases are sent to a phase separator or settling chamber for phase separation;
(j) the aqueous phase is recycled from the phase separator or settling chamber to the culture media reservoir or the bioreactor;
(k) the organic phase is sent to from the phase separator to a phase contactor or bubble chamber;
(l) the organic phase is enriched in oxygen or carbon dioxide in the phase contactor or bubble chamber;
(m) the enriched organic phase is recycled from the phase contractor or bubble chamber to the bioreactor.

35. The apparatus of claim 34 comprising:
(a) a plurality of process solution pumps separate from the bioreactor consisting of a culture media feed pump, a base pump, an acid pump, endotoxin neutralizing protein (ENP) pump, and antibiotic pump;
(b) a plurality of electromechanical solenoid process valves;
(c) a plurality of process heaters on the culture media reservoir and bioreactor or in the culture media reservoir and bioreactor;
(d) a plurality of gas tanks, gas regulators, and gas valves consisting of an oxygen tank, oxygen gas regulator, oxygen valve, carbon dioxide tank, carbon dioxide regulator, and carbon dioxide valve separate;
(e) a plurality of electrical (electronic) analog and digital process sensors consisting of temperature (thermocouple) sensors, hydrogen ion sensor, dissolved oxygen (DO) sensor, carbon dioxide (CO₂) sensor, dissolved nitric oxide (NO) sensor, endotoxin (E) sensor, antibiotic (A) sensor, and liquid level sensor;
(f) a plurality of microprocessor controllers accepting electrical (electronic) input signals (feedback) from process sensors generating electrical (electronic) output signals (feedback) to process pumps, process heaters consisting of a temperature (T) controller, hydrogen ion (pH) controller, dissolved oxygen (DO) controller, carbon dioxide (CO₂) controller, dissolved nitric oxide (NO) controller, endotoxin neutralizing protein (ENP) controller, antibiotic (A) controller, and liquid level controller;
(g) a data acquisition (DAQ) computer consisting of a keyboard, a pointing device (mouse), a graphical display (computer monitor), a hard drive (HD), random access memory (RAM), read only memory (ROM), erasable programmable read only memory (EPROM), and software program code separate from bioreactor accepting electrical (electronic) input signals (feedback) from process sensors and microprocessor controllers;
(h) a process control interface.

36. The apparatus of claim 34 wherein:
(a) the process pumps, process valves, process sensors, microprocessor controllers, and DAQ computer are electrically (electronically) interconnected with an analog and digital electrical (electronic) process control interface;
(b) cell culturing proceeds automatically while the bioreactor operates via process setpoints.

37. The apparatus of claim 34, wherein real time electrical (electronic) process data describing the chemical character of cell culture media during cell culturing is acquired and automatically recorded to a data file via data acquisition (DAQ) concurrent with cell culturing.

38. The apparatus of claim 34 wherein the cells are human cells, or transgenic mammalian cells, or non-transgenic mammalian cells, or transgenic porcine cells, or non-transgenic porcine cells, or transgenic animal cells, or non-transgenic animal cells, or transgenic fish cells.

39. The apparatus of claim 34 wherein:
(a) the organic phase is a liquid perfluorocarbon compound;
(b) the organic phase is hexadecafluoroheptane, or heptadecafluorooctane, or eicosafluorononane, or 1-bromotridecafluorohexane, or 1-bromoheptadecafluorooctane.

40. The apparatus of claim 34 wherein:
(a) the organic phase is a long chain aliphatic carbon compound;
(b) the organic phase is decane, or dodecane, or tridecane, tetradecane, or pentadecane, or hexadecane.
